(19) United States
(12) Patent Application Publication
(10) Pub. No.: US 2012/0203287 A1
(43) Pub. Date: Aug. 9, 2012

(54) MATERIAL DELIVERY APPARATUS FOR A BONE SCREW

(75) Inventors: Jared ARAMBULA, San Diego, CA (US); Sri VISHNUBHOTLA, San Diego, CA (US); Andrew MAHAR, San Diego, CA (US)

(73) Assignee: Alphatec Spine, Inc., Carlsbad, CA (US)

(21) Appl. No.: 13/364,173

(22) Filed: Feb. 1, 2012

Related U.S. Application Data
(60) Provisional application No. 61/439,165, filed on Feb. 3, 2011.

(51) Int. Cl. A61B 17/86; A61M 31/00

(52) U.S. Cl. 606/304; 604/48

(57) ABSTRACT

A material delivery apparatus for a bone screw includes an attachment member having a lumen disposed therethrough and adapted to removably attach to the bone screw. The apparatus further includes a delivery cannula fixedly disposed through the lumen and adapted to extend into a central bore of the bone screw for the delivery of material thereinto.

### CROSS-REFERENCE TO RELATED APPLICATIONS

This application claims priority to U.S. Provisional Application 61/439,165 filed Feb. 3, 2011, which is incorporated by reference herein in its entirety.

### FIELD

The present invention relates generally to an apparatus for delivery of material to a bone screw, and more specifically relates to an apparatus for sealed delivery of a material to an internal bore disposed longitudinally through a shaft of the bone screw.

### BACKGROUND

Certain spinal conditions, including a fracture of a vertebra and a herniated disc, indicate treatment by spinal immobilization. Several methods of spinal joint immobilization are known, including surgical fusion and the attachment of pins and bone plates via bone screws to the affected vertebrae. Often it may be desirable to introduce a material, for example, bone cement or a therapeutic material through the bone screw to the site of attachment. Prior apparatus for delivery of such material are known in the art.

One such known apparatus includes a fluid delivery probe that is removably attachable to a proximal portion of a pedicle screw via a snap fit secured by a nut against expansion or by a press fit backed by a set screw. An o-ring disposed on a distal end of the fluid delivery probe maintains a seal between the fluid delivery probe and a bore that extends through the shaft of the pedicle screw; however, a feeder tube connects a fluid source to a proximal end of the fluid delivery probe with no disclosure of a sealing or retaining connection mechanism connecting the feeder tube and the fluid delivery probe. Thus, the delivery of high pressure fluid through the connection poses the potential of a leak at the connection or the possibility of the feeder tube being blown off the fluid delivery probe by the high pressure fluid.

Moreover, the apparatus does not extend into the bore of the pedicle screw beyond the distal end of the fluid delivery probe. Thus fluid is delivered to a proximal end of the bore and must travel through the bore to get to discharge openings disposed through the shaft wall proximate a distal end of the bore. Fluid delivered only to the proximal end of the bore thus has a heightened chance of hardening before reaching the discharge openings or backing up proximally around the fluid delivery probe and causing a leak upstream of the bore due to internal pressure.

A second apparatus includes an elongate guiding portion that connects via threads, a press fit or a snap fit into a head of a bone screw including a bore through a shaft thereof. The second apparatus delivers material to a proximal end of the bore, thus also suffering the shortcomings of potential premature hardening and a heightened chance for leakage around the apparatus.

Thus, known apparatuses for delivery of a material to a bore in a shaft of a bone screw deliver the material to only a proximal end of the bore. A need exists for an apparatus that attaches to the bone screw and delivers material substantially beyond the proximal end of the bore.

### SUMMARY

In one aspect of the present invention, a material delivery apparatus for a bone screw includes an attachment member having a lumen disposed therethrough and adapted to removably attach to the bone screw. The apparatus further includes a delivery cannula fixedly disposed through the lumen and adapted to extend into a central bore of the bone screw for the delivery of material thereinto.

In another aspect of the present invention, a material delivery apparatus for a bone screw includes an attachment member having a lumen disposed therethrough and first threads disposed thereon adapted to engage second threads disposed on the bone screw. The apparatus further includes a delivery cannula fixedly disposed within the lumen and adapted to deliver material into a central bore of the bone screw. A transverse peripheral groove is disposed in an abluminal surface of the material delivery apparatus. The transverse peripheral groove is adapted to accommodate a sealing ring disposed within the groove.

In a further aspect of the present invention, a material delivery system for a bone screw includes an attachment member having a lumen disposed therethrough and first threads disposed thereon adapted to engage second threads disposed on the bone screw. The apparatus further includes a delivery cannula fixedly disposed through the lumen and adapted to extend into a central bore of the bone screw for the delivery of material thereinto. A transverse peripheral groove is disposed in an abluminal surface of the material delivery apparatus. The transverse peripheral groove is adapted to accommodate a sealing ring disposed within the groove. A material delivery source supplies material to the delivery cannula.

### BRIEF DESCRIPTION OF THE DRAWINGS

FIG. 1 illustrates an embodiment of a material delivery apparatus and a material source.

FIG. 2 is perspective view of the material delivery apparatus of FIG. 1.

FIG. 3 is a cross-sectional view of an embodiment of a material delivery apparatus taken generally along the lines 3-3 of FIG. 2.

FIG. 4 is a cross-sectional view of another embodiment of a material delivery apparatus taken generally along the lines 3-3 of FIG. 2.

FIG. 5 is a cross-sectional view of a further embodiment of a material delivery apparatus taken generally along the lines 3-3 of FIG. 2.

The foregoing and other features and advantages of the disclosure are apparent from the following detailed description of exemplary embodiments, read in conjunction with the accompanying drawings; wherein like structural or functional elements may be designated by like reference numerals.

### DETAILED DESCRIPTION

Disclosed is a material delivery apparatus able to delivery a viscous material into the cannulation of a bone screw. The instrument uses an attachment mechanism to removably affix to the head of the bone screw. The attachment mechanism uses an o-ring that is compressed during installation to the bone screw, plugging or sealing the cannulation of the bone screw, minimizing the possibility of expulsion of the viscous material during and after placement of the viscous material into the bone screw.

The material delivery apparatus is designed to deliver material substantially beyond a proximal end of a bore of a pedicle screw with a discharge opening proximate a distal end of the pedicle screw, which can lower the potential for premature hardening of the material before reaching the distal end of the pedicle screw. Such an apparatus also benefits from a lowered chance of the material backing up proximally around the apparatus and causing a leak upstream of the bore due to internal pressure. Further, such an apparatus including a cannula that extends substantially beyond the proximal end of the bore may benefit from application of a primary or secondary sealing ring to an abluminal surface of the cannula.

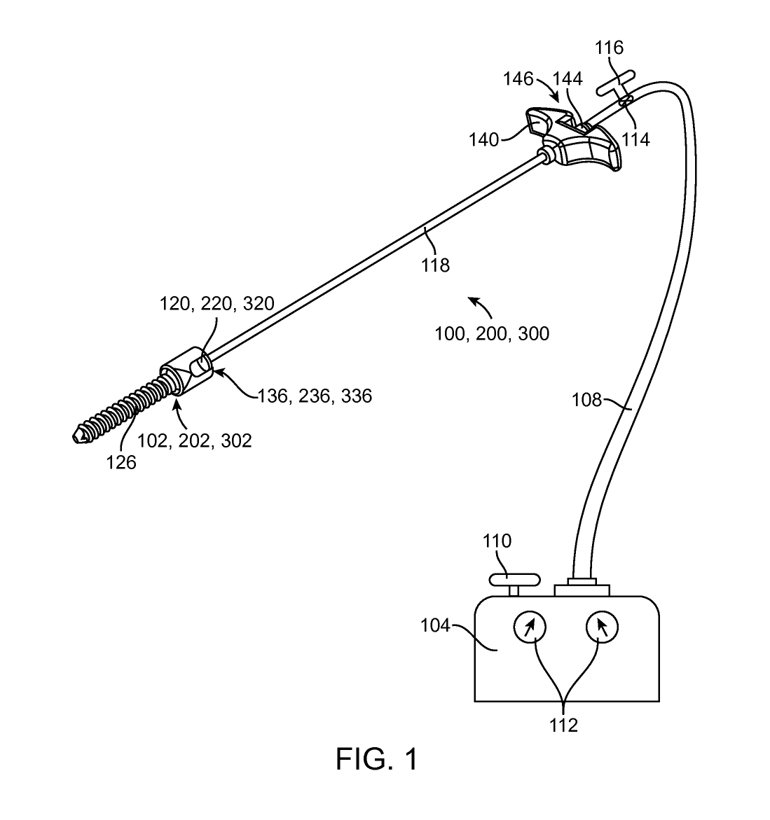

An embodiment of a material delivery apparatus 100 for a bone screw 102 is illustrated in FIG. 1. A source 104 of material may be connected to the apparatus 100 via a connector tube or hose 108. The source 104 may be pressurized and the material may comprise, for example, bone cement, a hardening polymer fluid, a therapeutic fluid, or other materials as known in the art. The tube 108 may be any suitable flexible tube or hose 108, including by way of example and not limitation, a low pressure tubing such as rubber or plastic tubing, or a high pressure tubing such as a steel braid reinforced hose, or any other suitable tubing as known in the art.

The source 104 may include a delivery valve 110, which in conjunction with pressure and flow rate gauges or indicators 112 may be used to meter the material at a desired rate and at a desired pressure, as known in the art. A second valve 114 proximate the apparatus 100 may allow a medical professional additional control over the flow rate and/or pressure of material delivery via a conveniently situated valve handle 116.

Referring to FIGS. 1-3, in one embodiment, the apparatus 100 includes a delivery cannula 118 and an attachment member 120 adapted to removably affix to the head or body member 136 of the bone screw 102 during material delivery. The delivery cannula 118 is fixedly disposed through a lumen 122 in the attachment member 120 and adapted to extend into a central bore 124 of a shaft 126 of the bone screw 102 for the delivery of the material (not shown) thereinto. The attachment member 120 may be attached to the delivery cannula 118, for example, via an adhesive, a fastener, a weld, or any other suitable method as known in the art. Alternatively, the attachment member 120 may be fabricated integrally with the delivery cannula 118. The delivery cannula 118 and the attachment member 120 may be made from any suitable material as known in the art including by way of example and not limitation, stainless steel, a thermoplastic, or other materials.

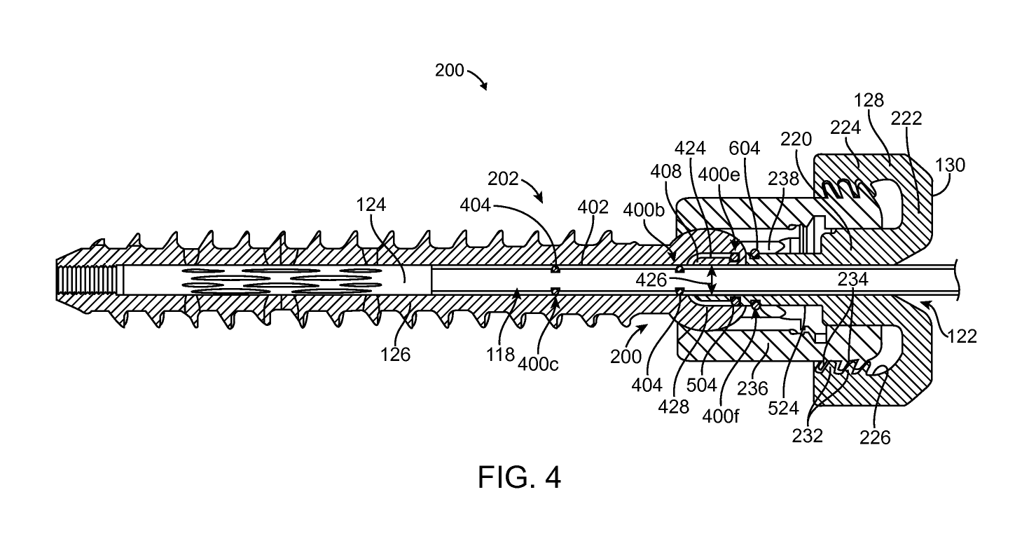

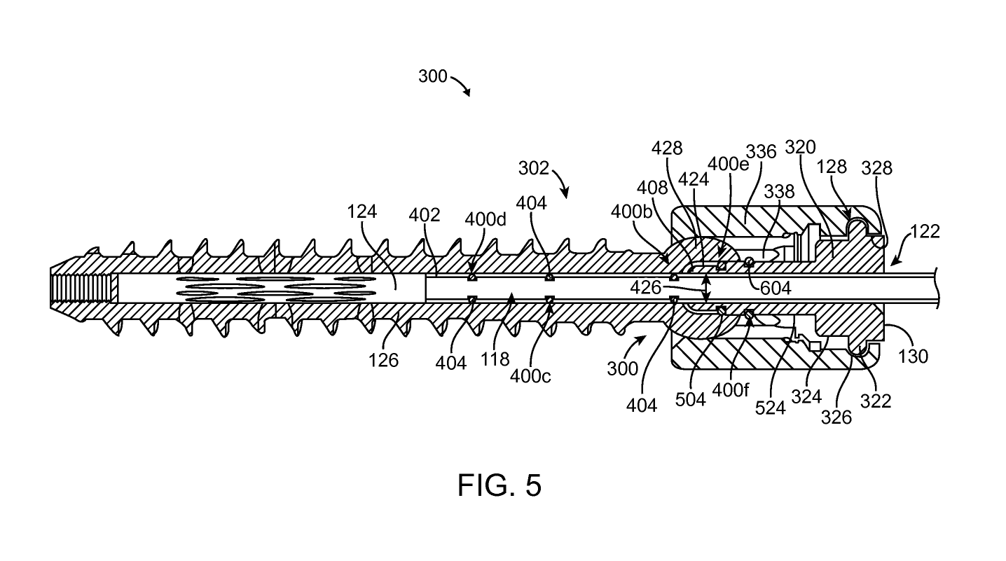

The attachment member 120 includes an attachment mechanism 128 proximate a proximal end 130 thereof. In one embodiment, the attachment mechanism 128 includes first threads 132 adapted to engage second threads 134 disposed on a body member 136 of the bone screw 102 for secure attachment. In this embodiment, the body member 136 has internal threads. In another embodiment of a material delivery apparatus 200, as shown in FIG. 4, an attachment member 220 includes a proximal flange 222 including an annular wall 224 extending distally therefrom. First threads 232 are disposed on a luminal surface 226 of the annular wall 224, as illustrated in FIG. 4. The first threads 232 are adapted to engage second threads 234 disposed on a body member 236 of a bone screw 202. In this embodiment, the body member 136 has external threads. In a further embodiment of a material delivery apparatus 300, an attachment member 320 includes a peripheral rib 322 extending from an outer peripheral or abluminal surface 324 thereof, as illustrated in FIG. 5. A groove 326 in a luminal surface 328 of a body member 336 of a bone screw 302 accommodates the peripheral rib 322 in a snap-fit arrangement. Further embodiments may include other suitable attachment mechanisms as known in the art for removably attaching the attachment member 120, 220, 320 to the body member 136, 236, 336, respectively.

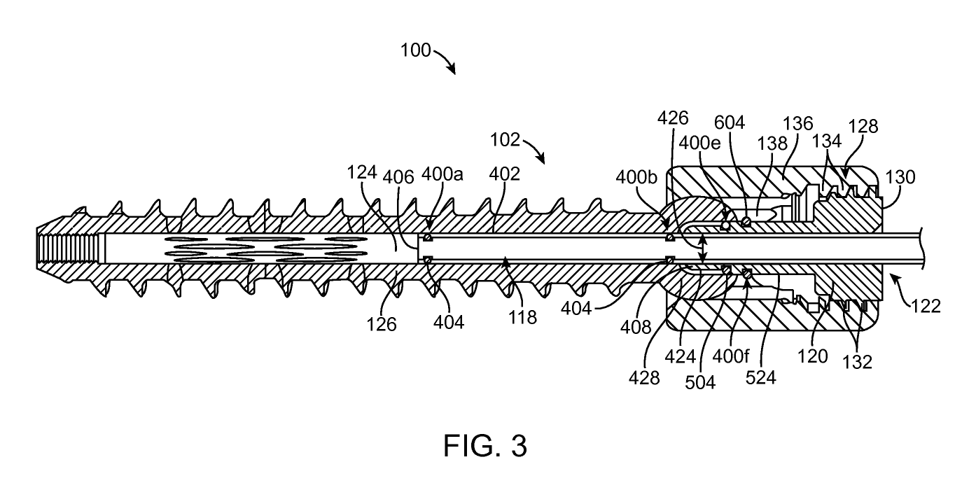

Referring to FIGS. 3-5, the delivery cannula 118 of any of the embodiments of the material delivery apparatus 100, 200, 300 disclosed hereinabove may include a transverse peripheral groove 400 disposed in an outer peripheral or abluminal surface 402 thereof. The transverse peripheral groove 400 is adapted to accommodate a sealing ring 404 disposed within the groove 400.

For example, a transverse peripheral groove 400a may be disposed proximate a distal end 406 of the delivery cannula 118, or a transverse peripheral groove 400b may be disposed proximate a distal end 408 of the attachment member 120, as illustrated in FIG. 3. In another embodiment, a transverse peripheral groove 400c may, for example, be disposed about half-way between the distal end 408 of the attachment member 120, 220, 320 and the distal end 406 of the delivery cannula 118, as illustrated in FIG. 4.

In other embodiments, a transverse peripheral groove 400d may be disposed at any axial location along the delivery cannula 118, for example, as illustrated in FIG. 5. In fact, any combination of the transverse peripheral grooves 400a-400d may be disposed along the delivery cannula 118 as may be appropriate. The transverse peripheral groove 400a-400d provides accommodation for the sealing ring 404 (shown in cross-section within each groove 400a-400d), which provides a seal between the abluminal surface 402 of the delivery cannula 118 and a luminal surface of the central bore 124.

In another embodiment, any of the attachment members 120, 220, 320 includes a transverse peripheral groove 400e disposed in an abluminal surface 424 thereof, which may or may not be coincident with the abluminal surface 324 described hereinabove with regard to FIG. 5. The abluminal surface 424 may have a larger radius than that of the abluminal surface 324, or the abluminal surface 424 may have a smaller radius than that of the abluminal surface 324, as illustrated in FIG. 5. The transverse peripheral groove 400e is adapted to accommodate a sealing ring 504 disposed within the peripheral groove 400e, as illustrated in FIGS. 3-5.

In one embodiment, the transverse peripheral groove 400e is disposed proximate the distal end 408 of the attachment member 120, 220, 320 at an axial position adapted to be disposed within a recess 426 in a head 428 of the bone screw 102, 202, 302, respectively, when the material delivery apparatus 100, 200, 300 is attached to the bone screw 102, 202, 302, respectively, as illustrated in FIGS. 3-5. Bone screws 102, 202, 302 that may be useful in the current invention include cannulated screws, fenestrated screws and expandable screws, for example, the expandable screws disclosed in Greenhalgh et al., U.S. Patent Application 2010/0228301, which is incorporated herein in its entirety. The sealing ring 504 provides a seal between the abluminal surface 424 of the attachment member 120, 220, 320 and a luminal surface of the recess 426.

In another embodiment, a peripheral groove 400f is disposed in an abluminal surface 524 of any of the attachment members 120, 220, 320. The abluminal surface 524 may be coincident with the abluminal surface 324 or the abluminal surface 424, both described hereinabove with regard to FIG. 5. Alternatively, the abluminal surface 524 may have a radius that is intermediate the radii of the abluminal surfaces 324, 424, as illustrated in FIG. 5. The transverse peripheral groove 400f is adapted to accommodate a sealing ring 604 disposed within the peripheral groove 400f. The transverse peripheral groove 400f is disposed at an axial position on the abluminal surface 524 that is adapted to be within the body member 136, 236, 336 of the bone screw 102, 202, 302, respectively, as illustrated in FIGS. 3-5.

In some embodiments, the body member 136, 236, 336 may include a bushing 138, 238, 338, for example, the bushing described in Purcell et al., U.S. Pat. No. 7,377,923, which is incorporated herein in its entirety. In those embodiments including the bushing 138, 238, 338, the sealing ring 604 provides a seal between the abluminal surface 524 of the attachment member 120, 220, 320 and a luminal surface of the bushing 138, 238, 338, respectively, as illustrated in FIGS. 3-5. In embodiments of the body member 136, 236, 336 that do not include a bushing, the sealing ring 604 provides a seal between the abluminal surface 524 of the attachment member 120, 220, 320 and a luminal surface of the body member 136, 236, 336, respectively.

Other embodiments of the attachment member 120, 220, 320 may include other transverse peripheral grooves (not shown) instead of or in addition to the transverse peripheral grooves 400e and 400f for the purpose of providing a seal between any of the outer peripheral surfaces 324, 424, 524 of any of the attachment members 120, 220, 320 and a luminal surface of any of the body members 136, 236, 336 or any of the bushings 138, 238, 338. In addition, one or more of the peripheral grooves 400a-400d disposed in the delivery cannula 118 as described hereinabove may be provided in combination with one or more of the peripheral grooves 400e-400g disposed in any of the attachment members 120, 220, 320.

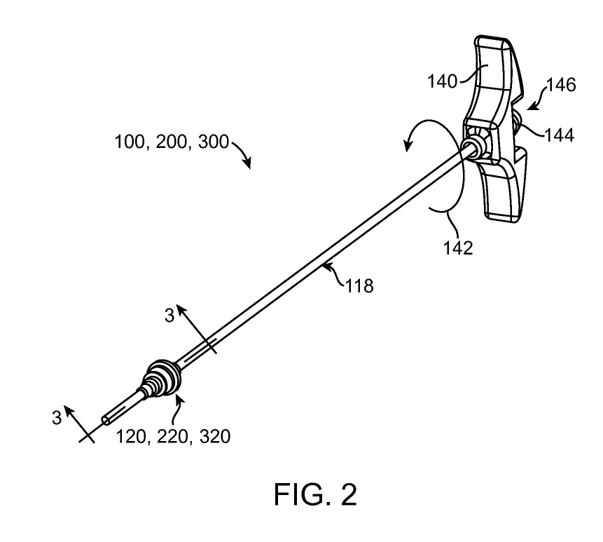

Any of the material delivery apparatus 100, 200, 300 may include components in addition to those described hereinabove. Referring to FIGS. 1 and 2, a handle 140 may be attached to the delivery cannula 118, for example, via an adhesive, a fastener, a weld, or any other suitable method as known in the art. Alternatively, the handle 140 may be fabricated integrally with the delivery cannula 118. The handle 140 and the delivery cannula 118 may be made from any suitable material as known in the art including by way of example and not limitation, stainless steel, a thermoplastic, or other materials. Rotation of the handle, as indicated by the arrow 142 illustrated in FIG. 2, rotates the delivery cannula 118 and the attachment member 120.

In addition to the handle 140, any of the material delivery apparatus 100, 200, 300 may further include a fluid fitting 144 attached to a proximal end 146 of the delivery cannula 118. The fluid fitting 144 may be any sort of fitting as known in the art and provides leak-free fluid communication between the delivery cannula 118 and the connector hose 108.

An improved material delivery apparatus for a bone screw is provided. The apparatus includes an extended cannula and is adapted to form a seal with one or more portions of the bone screw to inhibit leakage of material therefrom. The extended cannula is adapted to extend into a central bore of the bone screw to deliver material at a location substantially beyond the proximal end of the central bore. Such a delivery location benefits from a shorter travel path from the apparatus to discharge openings disposed through a shaft of the bone screw into the central bore, which can lower the potential for premature hardening of the material before reaching the discharge openings. Such delivery location also benefits from a lowered chance of the material backing up proximally around the apparatus and causing a leak upstream of the central bore due to internal pressure.

It will be appreciated by those skilled in the art that changes could be made to the embodiments described hereinabove without departing from the broad concepts disclosed therein. It is understood, therefore, that this disclosure is not limited to the particular embodiments disclosed, but it is intended to cover modifications that may include a combination of features illustrated in one or more embodiments with features illustrated in any other embodiments. Various modifications, equivalent processes, as well as numerous structures to which the present disclosure may be applicable will be readily apparent to those of skill in the art to which the present disclosure is directed upon review of the present specification. Accordingly, this description is to be construed as illustrative only and is presented for the purpose of enabling those skilled in the art to make and use the material delivery apparatus described herein and to teach the best mode of carrying out the same.

The invention claimed is:

1. A material delivery apparatus for a bone screw, comprising:
an attachment member having a lumen disposed therethrough and adapted to removably affix to the bone screw; and
a delivery cannula fixedly disposed through the lumen and adapted to extend into a central bore of the bone screw for the delivery of material thereinto.

2. The material delivery apparatus of claim 1, wherein the attachment member includes an attachment mechanism proximate a proximal end thereof.

3. The material delivery apparatus of claim 2, wherein the attachment mechanism comprises first threads adapted to engage second threads disposed on a body member of the bone screw.

4. The material delivery apparatus of claim 3, wherein the attachment member includes a proximal flange including an annular wall extending distally therefrom, and the first threads are disposed on a luminal surface of the annular wall.

5. The material delivery apparatus of claim 1, wherein the delivery cannula includes a transverse peripheral groove disposed in an abluminal surface thereof, wherein the transverse peripheral groove is adapted to accommodate a sealing ring disposed within the groove.

6. The material delivery apparatus of claim 1, wherein the attachment member includes a transverse peripheral groove disposed in an abluminal surface thereof, wherein the transverse peripheral groove is adapted to accommodate a sealing ring disposed within the groove.

7. The material delivery apparatus of claim 6, wherein the groove is disposed proximate a distal end of the attachment mechanism at a position adapted to be within a recess in a head of the bone screw when the material delivery apparatus is attached to the bone screw.

8. A material delivery apparatus for a bone screw, comprising:
an attachment member having a lumen disposed therethrough and first threads disposed thereon adapted to engage second threads disposed on the bone screw;
a delivery cannula fixedly disposed within the lumen and adapted to deliver material into a central bore of the bone screw; and
a transverse peripheral groove disposed in an abluminal surface of the material delivery apparatus, wherein the transverse peripheral groove is adapted to accommodate a sealing ring disposed within the groove.

9. The material delivery apparatus of claim 8, wherein the attachment member includes a proximal flange including an annular wall extending distally therefrom, and the first threads are disposed on a luminal surface of the annular wall.

10. The material delivery apparatus of claim 8, wherein transverse peripheral groove is disposed in an abluminal surface of the attachment member.

11. The material delivery apparatus of claim 10, wherein the transverse peripheral groove is disposed proximate a distal end of the attachment member at a position adapted to be within a recess in a head of the bone screw when the material delivery apparatus is attached to the bone screw.

12. The material delivery apparatus of claim 8, wherein the delivery cannula is fixedly disposed through the lumen and adapted to extend into a central bore of the bone screw for the delivery of material thereinto.

13. The material delivery apparatus of claim 12, wherein the transverse peripheral groove is disposed in an abluminal surface of the delivery cannula.

14. The material delivery apparatus of claim 13, wherein a second transverse peripheral groove is disposed in an abluminal surface of the attachment member, wherein the second transverse peripheral groove is adapted to accommodate a sealing ring disposed within the groove.

15. A material delivery system for a bone screw, comprising:
an attachment member having a lumen disposed therethrough and first threads disposed thereon adapted to engage second threads disposed on the bone screw;
a delivery cannula fixedly disposed through the lumen and adapted to extend into a central bore of the bone screw for the delivery of material thereinto;
a transverse peripheral groove disposed in an abluminal surface of the material delivery apparatus, wherein the transverse peripheral groove is adapted to accommodate a sealing ring disposed within the groove; and
a material delivery source that supplies material to the delivery cannula.

16. The material delivery system of claim 15, wherein the attachment member includes a proximal flange including an annular wall extending distally therefrom, and the first threads are disposed on a luminal surface of the annular wall.

17. The material delivery system of claim 15, wherein transverse peripheral groove is disposed in an abluminal surface of the delivery cannula.

18. The material delivery system of claim 15, wherein transverse peripheral groove is disposed in an abluminal surface of the attachment member.

19. The material delivery system of claim 15 further comprising a handle fixedly attached to the delivery cannula.

20. The material delivery system of claim 19 further comprising a fluid fitting attached to the delivery cannula proximal to the handle to control an amount of the material supplied by the material delivery source.
